(19) United States
(12) Patent Application Publication
(10) Pub. No.: US 2003/0045001 A1
(43) Pub. Date: Mar. 6, 2003

(54) Immunochromatographic test strip with arcuate sample application zone for ease-of-use in the field

(76) Inventors: Deborah Burgess, Mount Vernon, WA (US); Milton Tam, Seatle, WA (US); Ian Buchanan, Kirkland, WA (US)

Correspondence Address: Heller Ehrman White & McAuliffe LLP, 275 Middlefield Road, Menlo Park, CA, 94025-3506, US

(21) Appl. No.: 09/942,530

(22) Filed: Aug. 29, 2001

(51) Int. Cl. G01N 33/558

(52) U.S. Cl. 436/514

(57) ABSTRACT

A device, method of use and method of manufacturing of said device for detecting an analyte in a biological fluid. The device comprises an immunochromatographic test strip with a raised, arcuate sample receiving zone. The test strip comprises a bibulous strip positioned above the base wherein the bibulous strip defines a developer receiving zone, a sample application zone and an indicator zone and wherein the zones are in capillary contact with each other permitting a developing fluid to wick from the developer receiving zone, through the sample application zone to the indicator zone. Further, a mobilizable detector agent attached to a marker for binding to an analyte of interest in a sample is positioned downstream from the sample application zone and upstream from an immobilized indicator agent, which is attached within the indicator zone. The developing fluid transports the analyte of interest to a detector agent wherein the analyte is bound to the detector agent and is the complex is then transported to the indicator agent where it is bound again such that it produces a detectable signal. The sample application zone comprises of the invention an elevated region relative to the developer receiving zone and the indicator zone wherein the elevated region of the sample application zone can contain an observationally controllable volume of sample. The elevated sample application zone is arcuate in side profile and comprises lesser elevated regions contiguous with opaque borders residing both upstream and downstream from the elevated region.

### STATEMENT AS TO RIGHTS TO INVENTIONS MADE UNDER FEDERALLY SPONSORED RESEARCH AND DEVELOPMENT

This invention was made in part with governmental support under Cooperative Agreement Number HRN-A-00-96-90007 awarded by the Agency for International Development. The U.S. Government has certain rights in this invention.

### FIELD OF THE INVENTION

This invention relates to an improved immunochromatographic test strip comprising a distinctive raised, arcuate sample receiving area that allows for the direct application of a sample from the patient to the test strip in an observationally controllable way. The test strip comprises a sample application zone, an indicator zone and a developer receiving zone which are comprised of bibulous material and which are in fluid contact with each other. The invention also provides a method of determining the presence of an analyte in a biological sample and a method of manufacturing the improved test strip.

### BACKGROUND OF THE INVENTION

The relationship between the incidence of disease and poverty is well known. Epidemiology studies have shown conclusively that disease incidence and severity are significantly higher in the developing countries. Indeed, even in developed countries, the incidence of disease is higher in rural or poorer areas where access to healthcare services is problematic. Early diagnosis is often essential for effectively treating a disease. Many fatalities occur simply because the disease was not caught in an early enough stage for treatment to be effective. In many cases diseases go undiagnosed because of a lack of trained practitioners and adequate testing facilities. There continues to be a great need for diagnostic methods that are inexpensive and easy to use.

One effective tool for the detection of disease are test strips employing specific binding assays. The use of specific binding assays has been found to be of great value in a variety of clinical and other applications. Such assays involve the detection and determination of an analyte substance that is a member of a specific binding pair consisting of a ligand and a receptor. The ligand and the receptor are related in that the receptor specifically binds to the ligand, being capable of distinguishing a specific ligand or ligands from other sample constituents having similar characteristics. Immunological assays involving reactions between antibodies and antigens are one such example of a specific binding assay. Other examples include DNA and RNA hybridization reactions and binding reactions involving hormones and other biological receptors.

Specific binding assays may be practiced according to a variety of formats well known to the art. Such assays include competitive binding assays, “direct” and “indirect” sandwich assays and agglutination assays. Because the results of specific binding reactions are generally not directly observable, various techniques have been devised for labeling one member of the specific binding pair in order that the binding reaction may be indirectly observed. Useful labels include radiolabels, chromophores and fluorophores the presence of which may be detected by means of radiation detectors, spectrophotometers or visually determined by the naked eye. Where members of a specific binding pair are tagged with an enzyme label, their presence may be detected by the enzymatic activation of a reaction system including a signal generating substrate/cofactor group wherein a compound such as a dyestuff is activated to produce a detectable signal.

The use of reagent-impregnated test strips in specific binding assays such as immunoassays has previously been proposed. One such immunoassay carried out on a test strip involves lateral-flow chromatography. In this procedure, reagents are transported by a chromatographic solvent transport wherein a first reagent, is “picked-up” by the solvent as it flows via capillary action through bibulous material. The first reagent is often labeled with a molecule that can be observed. The labeling process may take place before the sample is added to the assay, i.e. it is admixed in a test tube and then applied to the sample receiving area, or perhaps the binding reagent has been labeled before. Thus, the labeled, bound analyte is transported to a second reagent that is immobilized where it then reacts to capture the analyte. Then, depending on the type of label, the presence of the analyte may be observed by whatever appropriate means are necessary to view the label.

Immunoassay procedures capable of being carried out on a test strip and which can be administered in the field or other locations where medical testing laboratories are not readily available have provided a great benefit to the diagnosis and control of disease. However, in many incidences these assays are not taken because there are not enough trained practitioners to administer them. Further, there remain some fundamental problems that make such a device impractical for use in the field. Immunochromatographic test strips often require collection of a biological fluid such as finger-stick blood. The biological fluid collected in the capillary tube must then be expelled onto a test strip and then the test run. It is often extremely difficult to effectively manipulate the capillary tube while at the same time holding the patient's finger and obtaining a sample of blood. Further, capillary tubes are often made of glass, thus presenting a safety hazard. These glass elements can also represent the most expensive component of the test kit. Further, the transfer of blood samples between testing components and the resulting bloodstained instruments increases the chance of infection to others who may come in contact with the blood.

In view of the above it was identified that if a biological sample could be directly applied to the test strip, it would eliminate the need for the capillary tube and would ultimately lower the end-cost of the test. Moreover, it would provide a sanitary and easy to use technique for remote testing. It was also determined that it is important to see and control the volume of blood being loaded onto the test strip so as to provide a means for determining whether there was a sufficient amount of the biological fluid obtained to conduct the test. It has been discovered that a raised sample application zone for a test strip would satisfy many of these needs.

There has been a large number of immunoassay devices disclosed over the last 20 years. Some immunoassays provide quantitative measurement of the amount of an analyte in a sample using complex, multi-step procedures and expensive analyzers that are available only in a laboratory setting. Other more simple immunochromatographic assays and the technology disclosed therein, such as those described in GB 2,204,398A; U.S. Pat. Nos. 5,096,837, 5,238,652, and 5,266,497; Birnbaum, S. et al., Analytical Biochem. 206:168-171 (1992); Roberts, M. A. and R. A. Durst, Analytical Chem. 67:482-491 (1995); and Klimov, A. D. et al., Clinical Chem. 41:1360 (1995), are incorporated herein in their entirety by reference.

However, none of these publications have disclosed a test strip comprising a raised, arcuate sample application zone. For example, the following patents are indicative of the general state of the art with regards to immunoassay test strips: U.S. Pat. No. 5,824,268 discloses a test strip, a device incorporating the test strip and a method for use of the test strip within the device for detecting an analyte in a solution or bodily fluid; U.S. Pat. No. 5,656,503 discloses a testing device using standard immunoassay techniques and reagents wherein a protruding sample pad is placed under a stream of sample fluid; and U.S. Pat. No. 5,620,845 recites an immunoassay process for detection of a immunologically active agent in a liquid sample employing a “dip-stick” type test strip.

The object of the present invention is, therefore, to provide a test device which is readily useable by an unskilled person and which allows the direct application of finger-stick blood or other biological samples to the test device in a controllable way, thus eliminating the need for a capillary-tube collection device or other collection or preparation procedures. The object of the inventions is satisfied by providing a lateral-flow chromatographic immunoassay test strip, which contains a raised, arcuate sample pad wherein a testing technician may simply apply the biological sample to the raised sample receiving area. For example, the technician could draw a blood droplet from a finger-stick, apply the sample pad of the test strip to the blood droplet until the pad is observably saturated, drop the strip into a developing agent and then read whether the analyte of interest is present or not by the presence (or absence) of a line or lines of color. Such is the ease of the present invention that a non-professional worker may easily be trained to administer these tests.

### SUMMARY OF THE INVENTION

The present invention is concerned with adapting and improving known diagnostic test devices wherein the improved test device allows direct application of a biological sample onto the device from the test subject.

In a first aspect, this invention provides an immunochromatographic test strip comprising a bibulous strip positioned above a base wherein the bibulous strip defines an upstream developer receiving zone, a downstream indicator zone and a sample application zone positioned between the upstream developer receiving zone and the downstream indicator zone. The zones are in capillary contact with each other thus permitting a developing fluid to wick from the developer receiving zone, through the sample application zone and then to the indicator zone. The sample application zone is elevated by the placement of a distinctive elevating member so that it extends above the plane of the test strip. Thus, a biological sample can be placed onto the elevated sample application zone such that the volume applied can be observed and controlled. A detector agent comprising a specific binding agent attached to a marker is positioned downstream from the sample application zone and upstream from the downstream indicator zone. Further, an analyte indicator agent that is specifically reactive with the same analyte as the detector agent is attached within the indicator zone. A developing fluid can then transport an analyte of interest from the biological sample on the sample application zone to the detector agent where the analyte (or analytes) of interest is bound. The detector agent/analyte complex is transported to the analyte indicator agent where the analyte is bound again, at a position distal from the detector agent. The attached marker of the detector agent provides a detectable signal at the location of the analyte indicator agent when the specific analyte is present.

In another preferred embodiment, the elevated sample application zone of the immunochromatographic test strip is distinctively arcuate in side profile and comprises lesser elevated regions contiguous with opaque borders residing both upstream and downstream from the elevated region.

In yet another preferred embodiment, the sample application zone of the immunochromatographic test strip comprises a cylindrical rod positioned between the base and the sample zone to elevate the surface of the sample zone.

In yet another preferred embodiment, the immunochromatographic test strip has opaque borders located on the lesser elevations of the elevated sample application zone wherein the ratio between the height of the elevation and the distance between the opaque borders is between 0.3:1 and 0.5:1.

In yet another preferred embodiment, the sample application zone of the immunochromatographic test strip provides a height of elevation from the lesser elevated regions of between 0.1 and 0.4 cm, and a distance between the opaque borders between 0.3 and 0.5 cm.

In a second aspect the invention provides a method of determining the presence of an analyte in a biological sample using the immunochromatographic test strip, the method comprising the steps of firstly providing a base and a bibulous strip located above the base wherein the bibulous strip defines an upstream developer receiving zone, a downstream indicator zone and a sample application zone positioned between the upstream developer receiving zone and the downstream indicator zone. The zones are in capillary contact with each other thus permitting a developing fluid to wick from the developer receiving zone, through the sample application zone and then to the indicator zone. The sample application zone is elevated by the placement of an elevating member so that it extends above the plane of the test strip. Secondly, the method providing a detector agent comprising a specific binding agent attached to a marker wherein the detector agent is positioned upstream form the sample application zone and downstream from the upstream indicator zone. Thirdly, the method providing an analyte indicator agent that is specifically reactive with the same analyte as the detector agent, wherein the analyte indicator agent is bound within the indicator zone. The method then requires the placing of a biological sample that possibly contains an analyte of interest onto the elevated sample application zone such that the amount applied can be observed and controlled. Finally, a developing fluid is applied to the developer receiving zone and wherein the developing fluid, through capillary migration, will wick through the test strip. The developing fluid transports the analyte of interest to the detector agent where it is bound. The detector agent/analyte complex is then transported to the analyte indicator agent where the analyte is bound again, at a position distal from the detection agent. The attached marker of the detector agent provides a detectable signal at the location of the analyte indicator agent when an analyte of interest is present.

In a third aspect, the present invention provides a method of manufacturing an immunochromatographic test strip having an elevated sample application zone. The method entails firstly providing a base and a bibulous strip located above the base wherein the bibulous strip defines an upstream developer receiving zone, and a downstream indicator zone. The zones are placed such that they remain in capillary contact with each other thus permitting a developing fluid to wick from the developer receiving zone, through the sample application zone and then to the indicator zone. Secondly, elevating the sample application zone is accomplished by the placement of an elevating member so that the application zone extends above the plane of the test strip. Thirdly, providing a detector agent wherein the detector agent comprises a specific binding agent that is reactive with the same specific analyte as the analyte indicator agent, where the analyte indicator agent is attached within the indicator zone.

In a fourth aspect, the present invention provides a diagnostic kit comprising the test strip of the subject invention and a vial, or a plurality of vials, having a removable cap. The kit will also include a container containing a developing solution and a packaged, sterile lancet.

In another preferred embodiment, the vial or vials of the diagnostic kit will contain a premeasured amount of developing fluid, wherein the fluid provided is of an amount sufficient to develop the test strip.

### BRIEF DESCRIPTION OF THE DRAWINGS

FIG. 1 is an exploded perspective view of an immunochromatographic assay device with an elevated, arcuate sample application zone in accordance with the invention;

FIG. 2 is a perspective view of an immunochromatographic assay device with a raised, arcuate sample application zone in accordance with the invention;

FIG. 3 is a cartoon depicting the application of an immunochromatographic assay device with a raised, arcuate sample application zone to a finger-prick blood sample, in accordance with the method of use of the invention; and

FIG. 4 is a cartoon depicting the application of an immunochromatographic assay device with a raised, arcuate sample application zone, which had been loaded with a blood sample, being placed into a test tube containing a developing fluid, in accordance with the method of use of the subject invention.

### DEFINITIONS

The term “analyte,” as used herein refers to the compound or composition to be detected that is capable of binding specifically to an antigen, an antibody, a receptor or other binding agent. Examples of analytes include monovalent analytes such as drugs, hormones, pesticides, organic chemicals and the like, as well as polyvalent analytes such as polypeptides and proteins including immunoglobulins, polysaccharides, nucleic acids, and combinations thereof. Such combinations or assemblages include bacteria, viruses, chromosomes, genes, mitochondria, nuclei, cell membranes and the like.

The term “antigen,” as used herein refers to all substances which in the form of a protein-complex, have antigenic activity, including high molecular weight proteinaceous components of human body liquids, hormones, bacteria, specific bound bacteria antigens, bacteria-produced toxins, virus etc., and lower molecular weight chemicals which react with a body protein to form a protein-complex with antigenic activity.

The term “antibody,” as used herein refers to a specific class of proteins (immunoglobulins) which exhibit a specific immunological activity against the antigen which caused their formation. Antibodies are very similar in their overall protein-structure, but are distinguished from each other by their specific affinity to different antigens. Antibodies are a proteinaceous material which exhibit antibody activity. That is, the antibody is a substance, in substantially pure form or in form of a mixture, having a high antibody-activity, e.g., an anti-serum. Antibodies can be an immunoglobulin or derivative or fragment thereof having an area on the surface or in a cavity, which specifically binds to and is thereby defined as complementary with a particular spatial and polar organization of another molecule. The antibody can be monoclonal or polyclonal, natural (native) or recombinant, including antibody fragments such as F(ab)′, F(ab)′₂ and scFv, can be prepared by techniques that are well known in the art such as immunization of a host and collection of sera or hybridoma technology.

The term “analyte binding reagent,” as used herein refers to any compound or composition capable of recognizing a particular spatial and polar organization of an analyte. In the case of an analyte which is a specific immunoglobulin species, the analyte-binding reagent may be the specific protein or epitope recognized by the immunoglobulin. Other types of analyte binding reagents include naturally occurring receptors, antibodies, enzymes, Fab fragments, lectins, nucleic acids, avidin, protein A, complement component Clq, and the like.

The term “detection agent,” as used herein refers to a material that is an analyte-binding reagent that is labeled with a substance capable of being detected. For an assay for specific forms of antigen, a preferred detection agent would be an antibody against the antigen, or a specific protein of that antigen, labeled with colloidal gold. For an assay for specific forms of antibody, a preferred detection reagent would be protein A labeled with colloidal gold. Other detection reagents for antibody as an analyte would include an antibody directed to the antibody that is the analyte, e.g., goat antihuman IgG labeled with gold for detection of human antibody. The detector agent may also be conjugated to hapten such as biotin and the detector agent may be specific for such hapten such as, e.g., antibiotin or avidin. Further, an anti-viral binding compound may be employed when the analyte is a virus.

The term “analyte indicator agent” as used herein also refers to any analyte binding reagent, but one that is not labeled. For an assay for specific forms of antigen, a preferred detection agent would be an antibody against the antigen, or a specific protein of that antigen. For an assay for specific forms of antibody, a preferred detection reagent would be protein A. Other detection reagents for antibody as an analyte would include an antibody directed to the antibody that is the analyte, e.g., goat antihuman IgG for detection of human antibody. Alternatively, indicator agent may be conjugated to hapten, such as biotin, and the detector agent may be specific for such hapten such as, e.g., antibiotin or avidin.

The term “marker” or “label” as used herein refers to any molecule bound or conjugated to an analyte binding reagent that can produce a signal. In the subject invention, the marker is capable of spontaneously producing a detectable signal without any additional reagents and will be detectable by visual means. The preferred embodiment is colloidal gold as a marker. Other embodiments could include other colloidal metals, colored particles, or liposomes filled with a colored substance. Many such substances will be known to those skilled in the art.

The term “bibulous” as used herein refers to a porous material having pores of at least 0.1 μ, preferably at least 1.0 μ, which is susceptible to traversal by an aqueous medium in response to capillary force. Such materials are generally hydrophilic or are capable of being rendered hydrophilic and include inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, polyvinyl chloride, polyacrylamide, cross-linked dextran, agarose, polyacrylate, glass fibers, polyesters, etc.; either used by themselves or in conjunction with other materials; ceramic materials; and the like.

The term “immunochromatographic test strip,” as used herein refers to bibulous material which contains analyte binding reagents, preferably antibody or antigen detection reagents that are arranged so that a sample fluid possibly containing an analyte can be applied and reacted with the reagents such that the presence of the analyte will be determined.

The term “opaque borders,” as used herein refers to visible markers that define an area or the circumference of an area where a sample fluid possibly containing an analyte will be applied. Thus, the opaque borders determine if a suitable amount of sample has been applied.

The term “capillary contact” as used herein refers to an overlap of or physical contact between at least two discrete portions of fibrous or bibulous material such that fluid may flow from one portion of material to the other.

### DETAILED DESCRIPTION

Introduction

The present invention is concerned with adapting and improving known diagnostic-test devices for the detection of an analyte in a biological fluid, which incorporate specific binding pairs. It has recently been discovered that a raised, arcuate sample application zone has special utility as a means for collecting and controlling the volume of a biological sample from a test subject. An immunochromatographic test strip employing such a sample collection area would be particularly beneficial to administering an immunoassay type test to a subject outside of a testing facility, i.e., the field. Principally, an immunoassay test strip device with a raised, arcuate sample zone would be exceptionally helpful in developing countries or rural areas in any country where access to medical facilities is limited.

The Test Strip

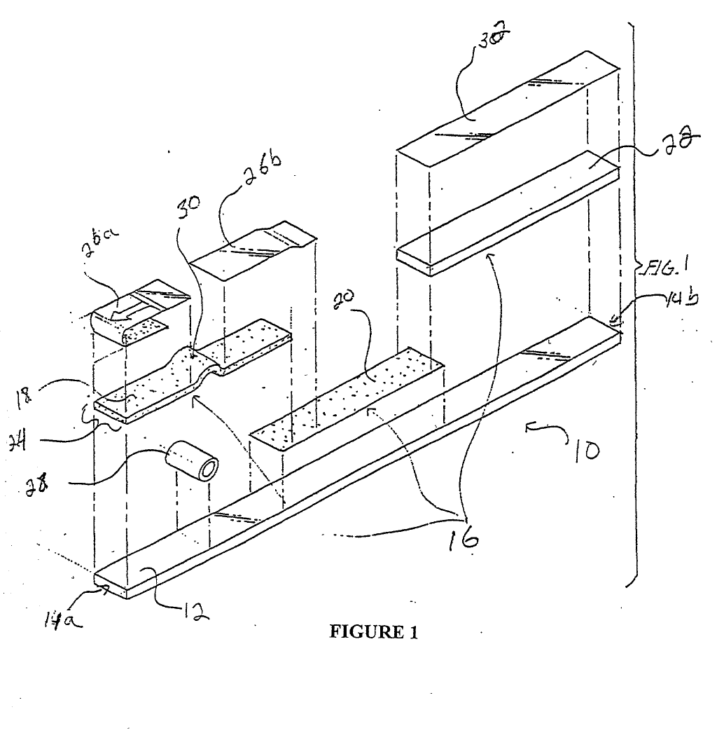

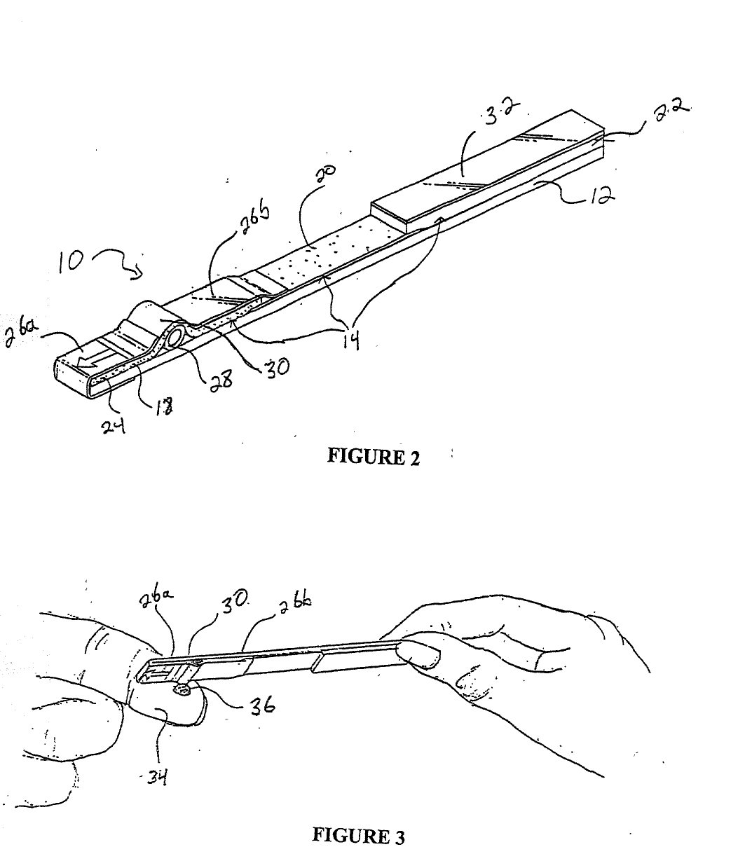

Referring now to the drawings, where like reference numerals identify like parts, there is illustrated FIG. 1 and FIG. 2, the immunochromatographic assay test strip in accordance with the invention, generally shown as 10. The immunochromatographic test strip 10 comprises a thin, rigid backing strip 12 which is simply an inert supporting base. Arranged on top of the backing strip 12 are the components of bibulous strip 16. As bibulous strip 16 is composed of a material capable of transporting a developing fluid laterally through capillary migration, the orientation of the components and features of the test strip are described as being upstream or down-stream in relation to other component and features, wherein upstream is defined as being toward beginning end 14a and downstream as in the direction toward terminal end 14b.

Bibulous strip 16 defines a sample application zone, an indicator zone and a wicking zone. Bibulous strip 16 can be a single piece of bibulous material, adhered to one surface (herein the top portion) of backing strip 12, and define all three zones. However, for ease of manufacture it is preferred that bibulous strip 16 comprise at least two discrete components of bibulous material in physical contact with each other. In that embodiment indicator strip 20 and wicking element 22 would comprise a single strip of bibulous material wherein the strip would be sufficiently long to provide the necessary fluidic draw of a wicking element and the sample application zone would comprise sample pad 18 as a different bibulous piece. Alternatively, sample application pad 18 and indicator strip 20 could be a single bibulous strip and wicking element 22 comprise a second bibulous component. In the most preferred embodiment, which is illustrated in FIGS. 1 and 2, bibulous strip 16 is comprised of three discrete components, all of which are in fluidic contact through capillary migration with each other. Thus, sample pad 18 defines a sample application zone, indicator strip 20 defines an indicator zone and wicking element 22 defines a wicking zone.

The immunochromatographic assay test strip 10 comprises a rectangular backing strip 12 to which sample application pad 18 is attached by a suitable adhesive. Sample application pad 18 is attached from the edge of beginning end 14a and extends about a third of the length of backing strip 12 in the downstream direction. Disposed between the central portion of sample application pad 18 and backing strip 12 is elevating member 28. Elevating member 28 elevates a portion of sample application pad 18 into a raised, arcuate shape and creates sample receiving area 30. The portion of sample application pad 18 that is upstream from sample receiving area 30 is defined as developer receiving zone 24. The downstream edge of sample application pad 18 slightly overlaps indicator strip 20, which is adhered along the central portion of backing strip 12. Wicking element 22 is adhered from the edge of terminal end 14b and extends back up backing strip 12 in the upstream direction, just slightly overlapping the downstream edge of indicator strip 20. Thus, sample application pad 18, indicator strip 20 and wicking element 22 lie end to end to form a bibulous strip over backing strip 12.

The invention takes advantage of masking strips to enhance its ease of use. Mask strip 26a is attached from the upstream lower elevation (the foot) of the raised, arcuate portion of sample receiving area 30 and extends to and wraps around beginning edge 14a so that it reverses direction and terminates 2 to 10 mm from beginning edge 14a, but on the bottom portion of backing strip 12. Masking strip 26b extends from the downstream foot of the raised, arcuate sample receiving area 30 to just over the downstream edge of sample application pad 18, contacting indicator strip 20, and covering the overlap of the two bibulous pieces. Thus, masking strips 26a and 26b cover sample application pad 18 except for the raised, arcuate sample receiving area 30, in essence defining opaque borders of sample receiving area 30. Further, mask strip 26a contains an orientation indicator which shows the user which end will be placed into the developing solution. Masking strip 32 covers the entire surface of the wicking element 22. Masking strip 32 comprises a white-matte plastic strip, preferably polyethylene, which is covered by a clear laminate. This strip provides a barrier between the wicking element, which can become saturated during the test, and the user's hand and further provides a means to imprint relevant test information, such as the type of test the strip is useful for as well as patient data.

Backing strip 12 will normally be a water insoluble, non-porous, and rigid material and usually will be of the same length and width as the bibulous strip but may be larger or smaller than the bibulous strip. A wide variety of organic and inorganic materials, both natural and synthetic, and combinations thereof, may be employed provided that the support does not interfere with the capillary action of the strip, or non-specifically bind assay components, or interfere with the signal producing system. Illustrative polymers that may be used include polyethylene, polypropylene, poly(4-methylbutene), polystyrene, polymethacrylate, poly(ethylene terephthalate), nylon, poly(vinyl butyrate), glass ceramics, metals, and the like.

Sample application pad 18 can be prepared from any insoluble, fibrous and absorbent material to which analyte-binding reagent (herein “the detector agent’) preparations such as antibodies or antigens can be administered and which does not contain functional groups which will interfere with the analyte binding reaction. Especially suitable are fibrous materials that will not chemically bind with analyte binding reagents and into which such binding reagents will rapidly diffuse during the flow of a developing fluid. For example, any hydrophilic bibulous material such as paper, sponge, felt, porous polymers, organic or inorganic, and the like. In a preferred embodiment, the sample application pad 18 is comprised of polyester fiber.

The thickness and degree of porosity of sample application pad 18 are not critical and can vary depending on the particular assay for which the lateral-flow immunochromatographic type test strip and/or diagnostic kit is intended. Typically, the sample application pad is generally of 10 mm or less in thickness, having a pore size generally in the range of from about 0.01 micron to about 2 microns, and preferably from about 0.20 to about 0.50 microns, but pads of a thickness to about one-quarter inch and/or having a pore size up to about 100 microns may be employed in certain applications.

Elevating member 28 can be separate or an integral part of backing strip 12. It can be of any water insoluble, non-porous, and rigid material which will not react with the contemplated detector, indicator, developer or control agents detailed herein or otherwise chemically hinder the action of the assay. That is, any organic and inorganic materials, both natural and synthetic, and combinations thereof, may be employed including polyethylene, polypropylene, poly(4-methylbutene), polystyrene, polymethacrylate, poly(ethylene terephthalate), nylon, poly(vinyl butyrate), glass ceramics, metals, and the like.

Elevating member 28 can be of any diameter sufficient to elevate sample application pad 18 to a required height and steepness sufficient to practice the subject invention. Further, elevating member 28 will be of a length equidistant with the width of test strip 10 but could be smaller or larger. In a preferred embodiment, the rod has a diameter of between 1 to 3 mm and an optimum diameter of 2 mm, and a length of between 3 to 10 mm, optimally 5 mm. Elevating member 28 may be of any shape, which would provide the necessary support and contour to sample receiving area 30. Thus, elevating member 28 can be square-shaped, triangular-shaped, star-shaped or any sort of polygonal shape. Further, elevating member 28 may be solid or hollow, or even comprised of more than one material wherein one material surrounds the other. If elevating member 28 is hollow, then the diameter of the solid portion must be such that elevating member 28 will flexibly maintain its shape under pressure, such as from a single finger pressing down on sample receiving area 18. In the preferred embodiment, elevating member 28 is a hollow spherical shape wherein the inner diameter is between 0.5 to 1.5 mm and optimally at 1.1 mm. In an alternative and less favored embodiment, elevating member 28 may not be employed and instead sample application pad 18 is dimensioned such that there is sufficient material available for folding the material upon itself to create an elevated sample receiving area 30 of the dimensions described herein.

The dimensions and characteristics of the underlying elevating member 28 and the sample application pad 18 define the shape and area of sample receiving area 30 and thus the surface area available to absorb the applied sample. In effect, the shape of sample receiving area 30 is arcuate in side profile wherein there is a rounded peak of a certain elevation and the slopes to either side of the peak are curved until they flatten along backing strip 12. Additionally, the arcuate side profile is preferably symmetrical around the peak. The height of the peak can be from 1 to 6 mm but preferably is between 1 to 4 mm and optimally 3 mm. The distance from the beginning of the elevation (a foot) on one side of the elevated area to the other may be between 0.25 to 1.0 cm and preferably between 0.6 and 0.8 cm, optimally 0.75 cm.

The sample receiving area 30 may more accurately be described with respect to the borders provided by masking strips 26a and 26b since these strips are substantially impermeable and would not easily absorb any sample constituents. Further, while masking strips 26a and 26b can extend slightly below or exactly at the point where the elevation become non-elevated, it is preferred that the masking strips extend slightly upslope from the non-elevated portions of elevated sample receiving area 30. That is, masking strips 26a and 26b terminate at a lesser elevation upstream and downstream from the elevated region, respectively. Thus, the surface area of sample receiving area 30 is defined by the height of the elevation, the width of sample application pad 18 and the distance between its lesser elevated regions contiguous with the opaque borders of masking strips 26a and 26b. The surface area of sample receiving area 30 and the thickness of sample application pad 18 define or limit the volume of sample which can be deposited onto test strip 10. As such, the dimensions of sample receiving area 30 can be configured to accept the specific range of volume of sample fluid necessary to perform the assay for which test strip 10 is used. In a preferred embodiment the width of sample receiving area 30 is between 0.3 to 0.9 cm from the upstream opaque border to the downstream opaque border. In a more preferred embodiment the width is between 0.5 to 0.7 cm. Further, the ratio between the height of sample receiving area 30 to the distance between the opaque borders is preferably between 0.2:1 to 0.7:1 and most preferably between 0.3:1 to 0.5:1.

The indicator strip 20 can be of any bibulous material to which an analyte-binding agent can be bound. Preferably the material of indicator strip 20 is nitrocellulose having a pore size of at least about 1 micron, even more preferably of greater than about 5 microns, and yet more preferably about 8-20 microns. Preferably, the nitrocellulose sheet is “backed”, e.g. with a plastic sheet, to increase its structural and handling strength. The actual pore size of the nitrocellulose when backed in this manner will tend to be smaller than that of the corresponding unbacked material. The nitrocellulose has considerable advantage over conventional strip materials, such as paper, because it has a natural ability to bind proteins without requiring prior sensitization. Specific binding reagents, such as immunoglobulins, can be applied directly to nitrocellulose and immobilized thereon. No chemical treatment is required which might interfere with the essential specific binding activity of the reagent.

Wicking element 22 can be of any absorbent, hydrophilic bibulous material as will provide the necessary wicking strength to draw the developing fluid with the molecular components of the assay from the developer receiving zone and through the sample and indicator zones. For example, materials such as paper, sponge, felt, porous polymers and the like may be used. In a preferred embodiment, the sample application pad 18 is comprised of absorbent filter paper. Further, the dimensions of wicking element 22 is most preferably of a width equal to backing strip 12, though wicking element 22 could be wider or thinner. The length and thickness of wicking element 22 may vary and is defined by its functionality. In an alternative embodiment, as described above, the bibulous material of indicator strip 20 may be extended to such a length as to provide the appropriate wicking draw to pull the developing fluid through the bibulous strip through capillary migration.

The spatial separation between the zones, and the flow rate characteristics of the porous carrier material can vary as is described herein, but will be selected to allow adequate reaction times during which the necessary specific binding can occur, and to allow the labeled detection agent in the sample application zone to dissolve or disperse in the liquid sample and migrate through the carrier. Further control over these parameters can be achieved by the incorporation of viscosity modifiers (e.g. sugars and modified cellulose) in the sample to slow down the reagent migration.

Assays

An important aspect of the present invention is the use of specific analyte binding reagents for the detection of an analyte. While there are variations of assays, preferably immunoassays, that may be used with the subject invention such as competitive binding assays and agglutination reactions, a preferred embodiment is that of a sandwich assay using a solid support, which is described herein. One of skill in the art should be able to deduce the application of other types of assays known in the art using the subject test strip.

A sandwich assay involves the use of two binding components for a single analyte of interest. The first analyte binding reagent is usually immobilized to a surface whereupon a sample containing an analyte of interest, which is reactive with the immobilized binding agent, is applied over that surface and the analyte becomes bound to the immobilized reagent. Then the second analyte binding reagent, which incorporates a detectable label or marker, is applied to the surface and binds the analyte at a point distal from the immobilized first binding agent, thus creating a sandwich. Thereafter, the analyte is detected via the attached marker and in some instances is quantified.

The present invention preferably uses a sandwich assay as above with a slight variation. Specifically, the present invention requires that a mobilizable analyte binding reagent to an analyte of interest, referred to as the detector agent, be applied to a portion of sample application pad 18 which is downstream from sample receiving area 30 and upstream from the analyte indicator agent. The detector agent is labeled with a convenient detectable label. An analyte indicator agent, comprising an immobilized binding agent, that is specifically reactive with the same analyte as the detector agent, but not cross reactive with the detector agent, is attached at roughly the center portion of indicator strip 20. A developing fluid is applied at an upstream position from sample receiving area 30, which is loaded with a sample, and wicks through the bibulous strip 16 through capillary migration towards wicking element 22. Therein, the developing fluid will have transported an analyte of interest in the sample to the labeled detector agent, where the analyte of interest is bound, and then further transports the analyte/detector agent complex to the immobilized indicator agent, where the analyte is bound again at a point distal from the detector agent. Thus, the analyte is sandwiched between the two agents with the labeled detector agent to the outside where the label can be observed to detect the presence of the analyte.

The subject invention contemplates a wide variety of detector agents and analyte indicator agents that may be employed, depending on the analyte of interest. These binding constituents can be categorized into subsets based upon their specific nature. A preferred embodiment for the test strip of the present invention is the use of antibodies for the detector agent and indicator agent when the analyte is an antigen. The antibodies would both bind the antigen but would be derived from different subsets of that antibody, such as IgG and IgM.

Alternatively, the analyte could be an antibody and the detector agent protein A, human antibody or like substance and the indicator agent is an anti-analyte antibody. Additionally, the analyte may be a virus and the detector agent an antiviral binding compound. Other binding agents and associated analytes would be obvious to one skilled in the art.

As stated herein, one member of a binding pair which is described as the analyte indicator agent is bound to a portion of the indicator strip 20 wherein it will bind the labeled detector agent/analyte complex in a concentrated way so as to produce a detectable signal. The preferred embodiment defines indicator strip 20 as being comprised of nitrocellulose paper and therefore requiring no additional chemical agents to bind the indicator agent. However, the analyte indicator agent may be applied to indicator strip 20 in a variety of ways known in the art. In general, a region or small area of the porous surface of indicator strip 20 becomes a solid phase capturing surface by immobilizing the analyte indicator agent directly onto the surface of a indicator strip 20 or by indirectly attaching the indicator agent onto capture particles (i.e., latex, glass) which are immobilized on the surface of a indicator strip 20. Direct immobilization of the analyte indicator agent to an indicator strip 20 or capture particles bound on the indicator strip 20 occurs through electrostatic interaction, (i.e., differences in ionic charge), hydrophobic interaction, or covalent binding. Where capture particles are used the immobilization of capture particles to indicator strip 20 can also occur through the same phenomena or through size exclusion, preventing migration of the particles through the pores or fibers of indicator strip 20. Furthermore, the analyte indicator agent can be coupled to indicator strip 20 by other techniques such as coupling antibodies, either polyclonal or monoclonal, or directly by the well-known glutaraldehyde or succinimide methods.

The amount of analyte indicator agent and detector agent that is attached to indicator strip 20 and sample application pad 18 respectively is important, but may vary depending on the effective amount required and the affinity thereof to the bibulous strips. These factor are, in turn, dependent on the immunological reaction involved. Preferably, to enhance the sensitivity of the test and intensity of color formation for detection of the presence of the target antigen, the bound indicator agent will be applied to only a relatively small portion of one surface of the indicator strip 20 in a controlled linear, dot-like array or other desired pattern by jet-type atomizer sprayer, needle-like dispersing applicators and the like which apply a desired quantity of material in a concentrated narrow band or array. Preferably the indicator agent and detector agent are applied via a line dispenser in a linear fashion. This technique is particularly advantageous for the analyte indicator agent because it will allow a solid band of labeled analyte detector agent to be captured, indicating a positive test. In addition, while concentration may vary, it is preferred to apply between 0.5 to 4.0 micrograms of indicator agent or detector agent per 1 cm of bibulous strip in a linear band width from about 0. 1 mm to about 2 mm, most preferably about 0.7 mm across the full width of indicator strip 20.

Specific binding reagents, such as immunoglobulins, can be applied directly to nitrocellulose and immobilized thereon. No chemical treatment is required which might interfere with the essential specific binding activity of the reagent. Unused binding sites on the nitrocellulose can be blocked if so desired using simple materials, such as polyvinyl alcohol. Thus, following the application of the analyte binding reagents to the indicator zone, the remainder of the indicator strip material could be treated to block any remaining binding sites elsewhere. Treatment with protein (e.g. bovine serum albumin or milk protein), or with polyvinyl alcohol or ethanolamine, or any combination of these agents can achieve blocking. However, in the preferred embodiment such blocking is not critical.

As stated herein, the detector agent employed for the subject invention will incorporate a label, which when bound in the indicator zones, indicates the presence of the analyte of interest. The label can be any entity, the presence of which can be readily detected. Preferably the label is a direct label, i.e. an entity which, in its natural state, is readily visible either to the naked eye, or with the aid of an optical filter and/or applied stimulation, e.g. UV light to promote fluorescence. For example, minute colored particles, such as dye sols, metallic sols, fluorophores, radiophores and colored latex particles, are very suitable. Of these options, colloidal gold particles are most preferred. Concentration of the label into a small zone or volume should give rise to a readily detectable signal, e.g. a strongly colored area. This can be visually evaluated, or detected by instruments if desired.

Indirect labels, such as enzymes, e.g. alkaline phosphatase and horseradish peroxidase can be used, but these usually require the addition of one or more developing reagents such as substrates before a visible signal can be detected. Hence these are less preferred. Such additional reagents can be incorporated in the porous solid phase material or indicator zone 20 or in sample application pad 18, such that they dissolve or disperse in the aqueous liquid sample. Alternatively, the developing reagents can be added to the sample before contact with the porous material or the porous material can be exposed to the developing reagents after the binding reaction has taken place.

Coupling of the label to the specific binding reagent may be accomplished by covalent bonding, if desired, or by hydrophobic bonding. Such techniques are commonplace in the art, and form no part of the present invention.

The immunochromatographic test strips of the subject invention optionally provides a control means that can convey an unrelated signal to the user to indicate that the device has successfully completed a test. The control means is provided by a procedural control agent that is admixed with the detector agent and deposited with the detector agent on sample application pad 18. As the assay proceeds, the procedural control agent is transported along with the detector agent. An immobilized binding partner, the control indicator agent, to the procedural control agent is located on indicator strip 20. The control indicator agent binds the procedural control agent in all circumstances and indicates to the test user that the developing solution front has passed the indicator agent. Thus, a double line in indicator strip 20 indicates that the analyte of interest was present, wherein the indicator agent bound the labeled detector agent/analyte complex and the procedural control agent had bound the control indicator agent. Alternatively, the control zone can contain an anhydrous reagent or indicator that, when moistened produces a color change or color formation, e.g. anhydrous copper sulfate which will turn blue when moistened by an aqueous sample.

If desired, a device according to the invention can incorporate two or more discrete bibulous strips, e.g. separate strips or sheets, each carrying mobile and immobilized reagents. These discrete strips can be arranged in parallel, for example, such that a single application of liquid sample to the device initiates sample flow in the bibulous strips simultaneously. The separate analytical results that can be determined in this way can be used as control results, or if different reagents are used on the different carriers, the simultaneous determination of a plurality of analytes in a single sample can be made. Alternatively, multiple samples can be applied individually to an array of carriers and analyzed simultaneously.

Methods of Manufacture

Referring to FIGS. 1 and 2, there is shown the orientation of the components used for fashioning the immunochromatographic test strip with an elevated sample receiving area of the present invention. In particular, test strip 10 is constructed using the components already described herein.

The manufacture of the subject test strip begins by adhering the components that comprise bibulous strip 16 to backing strip 12. Prior to the formation of the individual test strips, backing strip 12 comprises a flat, rigid rectangular sheet of material such as plastic. For purposes of illustration, the sheet is orientated such that the distance from one side of the sheet to the other (the width) is greater than the distance from top to bottom (the length). A piece of bibulous material, which shall comprise indicator strip 20, is adhered to the center portion of backing strip 12 in a lengthwise orientation such that there exists exposed or uncovered top and bottom portions of backing strip 12 which are of approximately equal area. Alternatively, indicator strip 20 may comprise that same center portion and the whole of the top portion when indicator strip 20 also comprises the wicking element. However, in the preferred embodiment a piece of absorbent material that is wicking element 22 is adhered to the top portion of backing strip 12, filling in that entire portion. Further, wicking element 22 may be dimensioned slightly larger from top to bottom than the exposed top portion area of backing strip 12 such that wicking element 22 will overlap indicator strip 20. The overlap shall be of a distance to allow fluid flow between the two pieces of bibulous material through capillary migration and is preferably between 1 to 10 mm and most preferably at about 2 mm.

Sample receiving area 30 is constructed by first adhering elevating member 28 lengthwise onto the exposed bottom portion of backing strip 12. Elevating member 28 is placed in the center portion of the exposed area and extends widthwise from one edge to the other. Thereafter, the bibulous piece that is sample application pad 18 is then adhered to the bottom portion of backing strip 12 such that it covers the entire exposed area and elevating member 28. Further, sample application pad 18 may also be dimensioned so that it will overlap onto indicator strip to allow fluidic flow between the two pieces of bibulous material through capillary migration and the overlap is preferably between 1 to 10 mm and most preferably at around 2 mm. The material of sample pad 18 is pressed tightly against elevating member 28 to ensure close contact with the elevating member and backing strip 12 and thus form the raised, arcuate shape of the sample receiving area 30.

The detector agent is labeled and, if so desired, admixed with the labeled procedural control agent and applied to sample application pad 20. The mixture is applied as a single line onto sample application pad 18 running parallel to sample receiving area 30, wherein it is disposed between the raised arcuate surface and indicator strip 20. The detector agent line and the area immediately surrounding it may be treated with a mixture of protein and organic reagents to facilitate the efficient release of the label. For example, in a preferred embodiment wherein the label is colloidal gold, sample application pad 18 may be treated with bovine serum albumin, lauryl ether, and polyvinylpyrrolidone at pH 7.5. In turn, the indicator agent is bound to indicator strip 20. Once again, the agent is applied so that it is parallel to sample receiving area 30 but applied to the center of the indicator strip 20. If a procedural control agent is used, a separate line of the control indicator agent which binds the procedural control agent is applied above the indicator agent line, in between the control agent line and wicking element 22. The control indicator agent can be applied at any point above the indicator line but preferably lies 4 to 8 mm above it. Alternatively, the detector agent and control agents may be deposited onto the bibulous material prior to adhering the bibulous strips onto backing strip 12.

Upon completion of the dosing of the subject test strip, the mask strips are be applied. Masking strips 26a is applied over the bottom edge of the sample application area 18 so that it wraps around the edge and adheres to the bottom of backing strip 12. The other edge of masking strip 26a is attached at the bottom, lower elevation of sample receiving area 30. Mask strip 26b is attached from the top lower elevation of sample receiving area 30 and extends over the overlap of sample application pad 18 and indicator strip 20. Mask strip 32 is applied over the whole of wicking element 22 and comprises a piece of white matte polyethylene with a clear laminate top portion.

The assembled sheet is then cut into strips wherein the cut is applied from top to bottom, thereby producing the finished immunochromatographic test strip with a raised, arcuate sample receiving area. The strips can be of any width or length suitable but are preferably between 3 to 8 mm in width and 6 to 12 cm in length. Most preferably, the strip is 5 mm wide and 8 cm long. There, the test strip may be packaged in a suitable material known in the art, such as foil laminate package with desiccant.

Method of Use

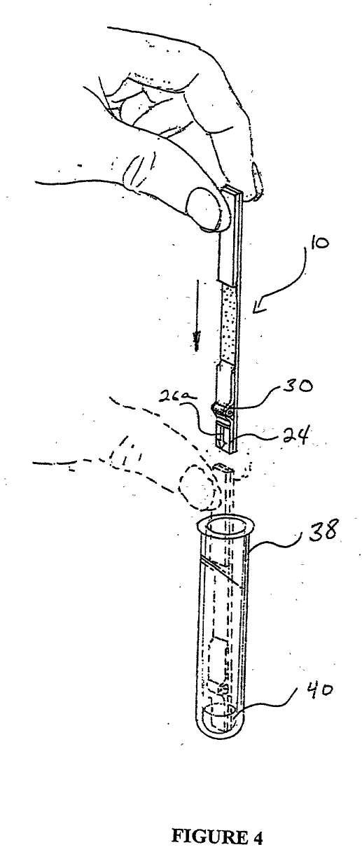

Referring now to FIGS. 3 and 4, which illustrate the process by which one may determine the presence of an analyte with the lateral-transfer immunochromatographic test strip with a raised, arcuate sample application zone. Specifically, FIG. 3 illustrates the application of the test strip as described above to a finger-prick blood sample. While the subject invention is not strictly limited to tests using a finger-stick blood sample, a preferred embodiment requires that the sample holder 34 is a finger and sample 36 is blood. Initially, the sample holder 34 is sterilized and lanced with a suitable device so that blood wells up to the surface of the skin. Test strip 10 of the subject invention is then applied to the blood droplets. This can be accomplished by turning the strip over such that the raised, arcuate sample receiving area 30 is facing downward toward the blood droplet or where the finger is orientated slightly downward and rolled across the elevated surface. Alternatively, the finger or any sample holder 34 can be held above sample receiving area 30 and the biological fluid allowed to flow out onto the raised area.

When contact is made between receiving area 30 and the blood droplet, the blood will be absorbed into test strip 10. Since sample receiving area 30 is elevated, sample holder 34 is disposed slightly off from test strip 10 while sample 36 is administered, thus leaving sample receiving area 30 exposed to visual inspection. In this manner, the volume of sample 36 applied is controllable by the test administrator. Since the volume of sample which can be applied is defined by the dimensions of sample receiving area 30 and the application of sample 36 is controlled by the test administrator, test strip 10 of the subject invention allows for a precise application of a volume of sample 36, thus ensuring that the correct volume of sample is deposited. Further, in the preferred embodiment wherein the elevated sample receiving area 30 is arcuate in side profile, the sides of sample receiving area 30 are exposed, which allows even greater control of the volume of sample applied. Generally, the blood should be allowed to wick into sample receiving area 30 until the test administrator can observe that the entire visible portion of the curved surface is saturated. In the preferred embodiment, saturation of the curved surface of the elevated sample application area 30 will result in approximately 5 to 10 μl of sample being drawn into the test strip. Oversaturation is not desired because it affects the readability of the test results.

FIG. 4 illustrates how the assay is performed after the collection of a sample. Test tube 38 contains chromatographic developing solution 40. Upon loading of a biological sample onto the elevated region of the sample receiving area 30, the immunochromatographic test strip 10 is placed into test tube 38 such that beginning end 14a enters the test tube 38 first. Preferably, masking strip 26a has imprinted upon its surface an arrow indicating the direction for which test strip 10 is inserted into test tube 38. Test tube 38 is configured such that test strip 10 is forced to remain substantially upright. Test strip 10 contacts developing solution 40 such that developer receiving zone 24 is completely immersed but developing solution 40 is not in contact with any portion of sample receiving area 30. Fluid contact with sample receiving area 30 would result in diffusion of the sample into developing fluid 40 which may result in an insufficient amount of analyte being available for detection. The test strip 10 is left in the developing solution 40 until a result is shown, preferably within 15 minutes.

While the test strip 10 is immersed in developing fluid 40, the fluid is being wicked into developer receiving zone 24 and migrates laterally towards the opposing end of test strip 10. Developing fluid 40 first contacts the sample loaded into sample receiving area 30 and carries the analyte to the region where the labeled detector agent had been deposited, just past the sample receiving area 30 on sample application pad 18. A binding reaction occurs if the analyte of interest is capable of being bound by the detector agent. While this reaction is occurring, developing fluid 40 continues to wick along the bibulous strip 16 towards the wicking element 22, transporting the analyte/labeled detector agent complex into the region of the immobilized analyte indicator agent on indicator strip 20. Upon contact with the immobilized analyte indicator agent, the analyte/labeled detector agent complex binds to the indicator agent, forming an aggregation of labels that can be visually observed.

As noted earlier, the test strip of the subject invention preferably uses a labeled procedural control agent, which is an mobilizable binding agent mixed and deposited with the labeled detector agent and a immobilized control indicator, which is a specific binding partner with procedural control agent and which is deposited just downstream of the analyte indicator agent deposition, preferably 4 to 16 mm. Thus, as the developing fluid wicks along the bibulous strip, it also transports the labeled procedural control agent over to the immobilized control indicator so that when the two agents bind, a second observable line forms. Therefore, formation of two lines on indicator strip 20, after running the immunochromatographic assay of the subject invention, would indicate a positive detection of the analyte of interest. A single line would indicate that the assay had been performed but that the analyte is not present, and no lines after a sufficient amount of time would indicate that the test failed and must be administered again.

Chromatographic developing solution 40 can be any solution known in the art capable of flowing through bibulous material. Further, the solution would be of a type that would transport the analyte and detector agent through the bibulous material without interfering or cross-reacting with the specific binding reactions contemplated. Thus, developing solution 40 may be any basic, acidic, polar, nonpolar, ionic or non-ionic fluid as is suitable for the molecular agents used and test strip components employed. In a preferred embodiment, wherein the biological sample is red blood cells, the developing fluid would be a buffer of a basic nature. The buffer lyses the red blood cells and frees the antigen into the plasma in addition to its transport capabilities. For example, the buffer may be Trizma® base. The amount of developing solution 40 employed would preferably be an amount sufficient to flow completely through the bibulous material and saturate wicking element 22, but must be at least 100 μl and preferably between 100 to 300 μl greater amounts are acceptable, as long as the sample is not leeched out.

Diagnostic Kit

The lateral-flow immunochromatographic test strip with an elevated, arcuate sample receiving area of the subject invention could easily be packaged as a diagnostic kit for use in the field. The kit would contain a test strip or a plurality of test strips packaged in foil-laminate containing a desiccant to preserve the test strip components. For example, the desiccant may be silica gel or molecular sieve. The test strip may, in turn, be housed in a cassette. The test strips will have been pre-fashioned with a specific binding pair for the specific analyte being tested for. A plurality of sealed test tubes or vials already loaded with the appropriate developing fluid at the optimal volume may also be supplied. A packaged lancet which is kept in a sterile condition, and a packaged sterile swab loaded with alcohol can be included. In an alternative embodiment, the sealed test tubes could be empty and the developing fluid provided in a separate container.

### EXAMPLES

The following examples are offered to illustrate, but not to limit the claimed invention.

Example 1 details the manufacture of the lateral-flow immunochromatographic test strip of the present invention.

Example 2 illustrates the utility of the lateral-flow immunochromatographic test strip with a raised, arcuate sample receiving area by testing for the presence of an analyte from a finger-prick blood sample.

### Example 1

Example 1 provides an illustration of one method of manufacturing a lateral-flow immunochromatographic test strip with a raised, arcuate sample application area.

The immunoassay device of the present invention was prepared by first adhering a 2.5×30.0 cm nitrocellulose (NC) paper strip of 10 μ-pore size, which was laminated to plastic backing, to a 8.0×30.0 cm white polystyrene kiss-cut card. The plastic-backed nitrocellulose was positioned lengthwise in the middle of white polystyrene kiss-cut card, leaving an area of 3.0×30.0 cm above the nitrocellulose strip and an area of 2.5×30.0 cm below the nitrocellulose strip.

A 30 cm length of nylon rod (Putnam Plastics, Corporation, Dayville, Conn.) with an internal diameter of 0.11 cm and an external diameter of 1.98 cm was first placed in the middle of the region below the nitrocellulose. A polyester-fiber material (Ahlstrom, Filtration Mount Holly Springs, Pa.) was attached over the nylon rod so that a platform was formed 3-4 mm above the plane of the test strip. The polyester-fiber material was placed so that a 2 mm overlap of bibulous materials was formed between the polyester-fiber material and the nitrocellulose. The polyester material was then firmly pushed onto the polyester card with sufficient force to ensure close contact over the nylon rod.

A 3.2×30 cm piece of absorbent filter paper (Schleicher and Schuell, Keene, N.H.), was then applied to the white polystyrene card in the region above the nitrocellulose. To ensure continuous capillary action, this absorbent filter paper was applied in such a way as to overlap onto the nitrocellulose by approximately 0.2 cm. Attachment of the plastic-backed nitrocellulose and the absorbent and polyester-fiber pads is facilitated through a layer of proprietary adhesive (G & L Die Precision, San Jose, Calif.) applied to the entire upper surface of the white polystyrene card.

A 3.0×30 cm white-matte polyethylene with a thickness of 0.076 cm and a 0.002 cm clear-over-laminate cosmetic mask was then placed over the absorbent pad in the region above the nitrocellulose so that the name of the test and the specimen/patient information could be written onto the test strip. Two cosmetic masks were then applied to the region below the nitrocellulose, above and below the raised platform. The first mask (1.4×30 cm) was positioned at the downstream foot of the raised platform, toward the nitrocellulose. The second mask (1.9×30 cm) was positioned at the upstream foot of the raised platform toward the edge of the test strip. The latter mask was wrapped underneath the base of the polystyrene card to about 7 mm to firmly secure the polyester-fiber material to the polystyrene card. Thus, the formation of an exposed area of polyester fiber over the nylon rod that can accommodate between 3-10 μl of finger-stick blood was created.

The assembled devices were then cut into 5 mm individual test strips and packaged in foil-laminate envelopes containing silica gel or molecular sieve desiccant. These test strips were stable at ambient temperature for many months and the test strip were still functional after that time.

### Example 2

Example 2 provides an example of the use of the immunochromatographic test strip of the present invention. The test strips are manufactured as detailed in Example 1, including a control agent, detector agent and indicator agent. The test strip is utilized for testing for the presence of an antigen derived from or an antibody specifically reactive to blood parasites, Tuberculosis, Dengue, Human Immunodeficiency virus, Hepatitis B virus, Hepatitis C virus, Syphilis, Chlamydia, Lyme disease, Staphylococcus or Pseudomonas in a sample population. The test is carried out over a period of several months in a sample population consisting of over 250 individuals. The finger from which the blood sample is to be drawn is first cleaned with antiseptic and allowed to air dry. A sterile lancet is used to prick the side of the finger and the released blood is directly applied to the raised collection area of the test strips. Blood is collected until the raised area on the test strip is saturated with blood and the test strip is then dropped into a provided plastic test tube containing 4 drops (approximately 100 μl) of a reaction buffer. The immunoassay strips are processed for about 30 minutes or until the control line is visible. The presence of the antigen or antibody of interest is indicated by observable lines across the indicator strip in the middle of the test strip. The development of two red lines on the test strip indicates a positive result, while one line would indicate a negative result. If neither the test line nor the procedural control line appears on the indicator zone, the test is deemed non-functional and is repeated.

Although the foregoing invention has been described in some detail by way of illustration and example, for the purposes of clarity and understanding it will be obvious that certain changes and modifications may be practiced within the scope of the claims. That is, those skilled in the art will recognize, or be able to ascertain using no more than routine experimentation, many equivalents to the specific embodiments of the invention described herein. Thus, such equivalents are intended to be encompassed in the scope of the following claims.

What is claimed is:

1. An immunochromatographic test strip comprising:
a base;
a bibulous strip positioned above the base; the bibulous strip comprising:
an upstream developer receiving zone;
a downstream indicator zone; and
a sample application zone between the upstream developer zone and the downstream indicator zone;
the zones in capillary contact with each other permitting a developing fluid to wick from the upstream developer receiving zone, through the sample application zone to the downstream indicator zone;
the downstream indicator zone containing analyte indicator agent for indicating the presence of an analyte of interest in the sample;
a detector agent for binding analyte of interest in the sample comprising a binding agent attached to a marker;
the sample application zone comprising an elevated region relative to the upstream developer receiving zone and the downstream indicator zone, the elevated region of the sample application zone for containing an observationally controllable volume of sample possibly having the analyte of interest;
the upstream developer receiving zone for receiving the developing fluid and permitting the developing fluid to wick through the sample application zone transporting the analyte of interest in the sample to the detector agent to form a detector agent/sample complex, then transporting the detector agent/sample complex to the analyte indicator agent in the downstream indicator zone to indicate through the marker the presence in the sample of the analyte of interest.

2. A test strip of claim 1 wherein the elevated region is arcuate in side profile.

3. A test strip of claim 2 wherein the elevated region has a symmetrical arcuate profile.

4. A test strip of claim 1 wherein the elevated region of the sample application zone further comprises lesser elevated regions contiguous with opaque borders residing both upstream and downstream from the elevated region.

5. A test strip of claim 4 wherein the ratio between the height of the elevation and the distance between the opaque borders is between 0.3:1 and 0.5:1.

6. A test strip of claim 4 wherein the height of the elevation from the lesser elevated regions is between 0.1 and 0.4 cm, and the distance between the opaque borders is between 0.3 and 0.5 cm.

7. A test strip of claim 1 wherein a cylindrical rod is positioned between the base and the sample application zone to elevate the surface of the sample application zone.

8. A test strip of claim 1 further comprising a procedural control agent wherein the procedural control agent is not cross-reactive with the analyte of interest, the detector agent or the indicator agent and is positioned downstream from the upstream developer receiving zone and upstream from the downstream indicator zone.

9. A test strip of claim 8 wherein the indicator zone further comprises a control indicator agent which binds only to the procedural control agent and is positioned downstream from the procedural control agent.

10. A test strip of claim 1 wherein the developing fluid is a buffer of a basic nature.

11. A test strip of claim 1 wherein the analyte of interest is an antigen derived from or an antibody specifically reactive to a member selected from the group consisting of: blood parasites, Tuberculosis, Dengue, Human Immunodeficiency virus, Hepatitis B virus, Hepatitis C virus, Syphilis, Chlamydia, Lyme disease, Staphylococcus and Pseudomonas.

12. A test strip of claim 1 wherein the marker is selected from a group consisting of a colloidal metal, colored particles, a liposome filled with a colored substance, an enzyme, a radiolabel, chromophore and a flourophore.

13. A test strip of claim 12 wherein the marker is colloidal gold.

14. A test strip of claim 1 wherein the sample is blood or plasma.

15. A method of determining the presence of an analyte in a biological sample utilizing an immunochromatographic test strip comprising the steps of:
providing a base;
providing a bibulous strip positioned above the base; the bibulous strip defining:
an upstream developer receiving zone;
a downstream indicator zone;
a sample application zone between the upstream developer zone and the downstream indicator zone, the sample application zone comprising an elevated region relative to the upstream developer receiving zone and the downstream indicator zone, the elevated region of the sample application zone for containing an observationally controllable volume of a biological sample possibly having an analyte of interest;
the zones in capillary contact with each other permitting a developing fluid to wick from the upstream developer receiving zone, through the sample application zone to the downstream indicator zone;
providing an analyte indicator agent for indicating the presence of the analyte of interest in the sample;
providing a detector agent for binding to the analyte of interest in the biological sample comprising a binding agent attached to a marker;
placing the detector agent downstream from the sample application zone and upstream from the analyte indicator agent;
applying the sample possibly having the analyte of interest to the sample application zone at the elevated region in an observationally controllable way;
applying developing fluid to the upstream developer receiving zone to permit the developing fluid to wick downstream through the sample application zone, transporting the analyte of interest in the biological sample to the detector agent to form a detector agent/analyte complex, then transporting the detector agent/sample complex to the analyte indicator agent in the downstream indicator zone to indicate through the marker the presence in the sample of the analyte of interest.

16. A method of claim 15 wherein the sample is blood or plasma.

17. A method of claim 15 wherein the sample is derived from a finger-stick or venipuncture.

18. A method of claim 15 further comprising providing a procedural control agent downstream from the upstream developer receiving zone and upstream from the downstream indicator zone, wherein the procedural control agent is not cross-reactive with the analyte of interest, the detector agent or the analyte indicator agent.

19. A method of claim 18 further comprising depositing a control indicator agent downstream from the procedural control agent wherein the control indicator agent binds only to the procedural control agent.

20. The method of claim 15 wherein the developing fluid is a buffer of a basic nature.

21. The method of claim 15 wherein said analyte is an antigen derived from or an antibody specifically reactive to a member selected from the group consisting of: blood parasites, Tuberculosis, Dengue, Human Immunodeficiency Virus, Hepatitis B Virus, Hepatitis C Virus, Syphilis, Chlamydia, Lyme Disease, Staphylococcus and Pseudomonas.

22. A method of manufacturing an immunochromatographic test strip comprising the steps of:
providing a base;
providing a bibulous strip positioned above the base; the bibulous strip defining:
an upstream developer receiving zone;
a downstream indicator zone; and
a sample application zone positioned between the upstream developer receiving zone and the downstream indicator zone, the sample application zone having an elevated region relative to the upstream developer receiving zone and the downstream indicator zone, the elevated region of the sample application zone for containing an observationally controllable volume of a sample possibly having an analyte of interest;
the zones in capillary contact with each other permitting a buffer to wick from the upstream developer receiving zone, through the sample application zone to the downstream indicator zone;
providing an analyte indicator agent in the downstream indicator zone for indicating the presence of the analyte of interest in the sample;
providing a detector agent for binding to an analyte of interest in a sample comprising a binding agent attached to a marker;
placing the detector agent downstream from the sample application zone and upstream from the analyte indicator agent;
elevating the sample application zone to define an elevated region relative to the upstream developer receiving zone and the downstream indicator zone, the elevated region of the sample application zone for containing an observationally controllable volume of sample possibly having an analyte of interest;
whereby the upstream developer receiving zone can receive developing fluid and permit the developing fluid to wick through the sample application zone, transporting the analyte of interest in the sample to the detector agent to form a detector agent/analyte complex, then transporting the detector agent/sample complex to the analyte indicator agent in the downstream indicator zone to indicate through the marker the presence in the sample of the analyte of interest.

23. A method of claim 22 further comprising providing a procedural control agent downstream from the upstream developer receiving zone and upstream from the downstream indicator zone, wherein the procedural control agent is not cross-reactive with the analyte of interest, the detector agent or the analyte indicator agent.

24. A method of claim 22 further comprising providing a control indicator agent which binds only to the procedural control agent and is positioned downstream from the procedural control agent.

25. A method of claim 22 wherein the elevated region is arcuate in side profile.

26. A method of claim 22 wherein the elevated region has a symmetrical arcuate profile.

27. A method of claim 22 wherein the elevated region of the sample application zone further comprises lesser elevated regions contiguous with opaque borders residing both upstream and downstream from the elevated regions.

28. A method of claim 27 wherein the ratio between the height of the elevation and the distance between the opaque borders is between 0.3:1 and 0.5:1.

29. A method of claim 27 wherein the height of the elevation from the lesser elevated regions is between 0.1 and 0.4 cm, and the distance between the opaque borders is between 0.3 and 0.5 cm.

30. A diagnostic kit comprising:
the test strip of claim 1;
a vial with a removable cap;
a container containing a developing solution; and a sterile lancet.

31. A diagnostic kit of claim 30 further comprising a plurality of vials, each with removable caps.

32. A diagnostic kit of claim 30 or 31 wherein the vial already contains a pre-measured amount of developing fluid sufficient to develop the test strip.

33. A immunochromatographic test strip comprising:
a base and a bibulous strip positioned above the base wherein the bibulous strip comprises a sample application zone and a test zone; wherein the two zones are in capillary contact with each other permitting fluid to flow between them, wherein the application zone is positioned upstream from the test zone, and wherein the application zone comprises an elevated region and lesser elevated regions contiguous with opaque borders residing both upstream and downstream from the elevated region.

34. A immunochromatographic test strip having a shape and volume of the sample application zone permitting a predictable absorption of a sample volume without requiring pre-measurement of the sample for use in a test requiring an accurate volume of test sample.
